(19) United States
(12) Patent Application Publication
(10) Pub. No.: US 2014/0052008 A1
(43) Pub. Date: Feb. 20, 2014

(54) HYPERTENSION MONITORING AND NOTIFICATION DEVICE BASED ON CONTEXT INFORMATION

(71) Applicants: Jong Hun Kim, Incheon (KR); Young Ho Lee, Gyeonggi-do (KR); Dong Kyun Park, Incheon (KR); Eun Young Jung, Incheon (KR)

(72) Inventors: Jong Hun Kim, Incheon (KR); Young Ho Lee, Gyeonggi-do (KR); Dong Kyun Park, Incheon (KR); Eun Young Jung, Incheon (KR)

(73) Assignees: GIL MEDICAL CENTER, Incheon (KR); GACHON UNIVERSITY OF MEDICINE & SCIENCE INDUSTRY- ACADEMIC COOPERATION FOUNDATION, Incheon (KR)

(21) Appl. No.: 14/006,270

(22) PCT Filed: Aug. 12, 2011
(86) PCT No.: PCT/KR2011/005957

§ 371 Date: Oct. 30, 2013

(30) Foreign Application Priority Data
Apr. 26, 2011 (KR) 10-2011-0038840

(51) Int. Cl. A61B 5/00; A61B 5/021

(52) U.S. Cl. CPC A61B 5/4866; A61B 5/021; A61B 5/7278; A61B 5/742; A61B 5/7246; A61B 5/0002
USPC 600/485

(57) ABSTRACT

Disclosed is a hypertension monitoring and notification device based on context information, which includes a data gain and storage unit for storing bio information of a user, context data, and weather and health information; a user context analysis unit for deducing context information of the user from the bio information and the context data to analyze context necessary for measuring hypertension; a specific hypertension analysis unit for analyzing specific hypertension using the context information; a blood pressure grade analysis unit for determining a blood pressure grade via the analyzed context information to deduce blood pressure signal light information and to transmit a recommendation content; and a result output and notification unit for outputting and notifying the user and the doctor of the bio information, the blood pressure signal light information, the specific hypertension information, and the context information.

### TECHNICAL FIELD

The present invention relates to a hypertension monitoring and notification device, and, more particularly, to a hypertension monitoring and notification device based on context information, which provides measurement and specific patient notification services linked with context information from the bio signal data of a user, and also provides a notification service depending on a variety of life indexes.

### BACKGROUND ART

With the recent trend of an aging population and an increase in a number of patients with chronic diseases, there is a heightened interest in employing health with IT technology, such that services related to health management are also increasing. Thereby, health conditions must be monitored and specialized health care services are required not only in hospitals but also in the patient's own home.

Hypertension, a chronic disease, is known to be an important risk factor for cerebrovascular and cardiovascular diseases which accounts for the second and third highest cause of death in Korea. Hypertension is typically caused by lifestyles and environmental factors such as obesity, smoking, high intake of salt, low potassium and magnesium intake, excessive intake of alcohol, stress, etc., resulting from a high-fat diet and from reduced exercise. The risk of hypertension is also increased by chronic diseases such as high cholesterol, diabetes, renal diseases, etc. In order to prevent and treat hypertension, day by day symptoms and improvements and the occurrence of complications are observed and recorded and reported to doctors so that accurate diagnosis and treatment are achieved. Furthermore, patients have to take a clear stand on their own to care for their diseases at home including taking medication or correcting their living habits.

With the goal of effectively carrying out and caring for their own diseases at home, ubiquitous-based IT technology is required more than anything else. Most services are focused on typical health care services based on remote controlled monitoring or merely providing a display of the measured bio signal information on a screen.

Such a system produces and manages information from the point of view not of users but of service providers. Furthermore, a lot of health information retrieved on the Internet may be easily utilized, but sites for providing such information and monitoring health conditions provide the measurer with limited measurement results based on the daily measurement numerals and prescriptions only based on the results, making it difficult to continuously manage the health of patients.

Like this, conventional monitoring services provide only prescriptions based on measurement results without grasping the measurement context of a user, and the provided health information or other contents are not customized to the user, making it difficult to continuously manage the health of patients.

### DISCLOSURE OF INVENTION

### Technical Problem

Accordingly, the present invention has been made keeping in mind of the above problems occurring in the related art, and an object of the present invention is to provide a hypertension monitoring and notification device, which provides context information when taking blood pressure measurements of a patient with hypertension so that the patient can be continuously cared for at home and a doctor clinically uses the data measured at the home, by which the context is analyzed so that the user complies with measurement suggestions, and recommendation and notification services are provided, which also enables the classification of a patient suspected of having specific hypertension who is difficult to diagnose in a hospital, so as to notify a doctor of the status.

### Solution to Problem

In order to accomplish the above objects, an aspect of the present invention provides a hypertension monitoring and notification device based on context information, comprising a data gain and storage unit for collecting bio information of a user and context data, and storing weather and health information; a user context analysis unit for deducing context information of the user from the bio information and the context data to analyze context necessary to measure hypertension; a specific hypertension analysis unit for analyzing specific hypertension using the bio information and the context information; a blood pressure grade analysis unit for enabling a doctor to determine a blood pressure grade via the analyzed context information so that blood pressure signal light information is deduced and a recommendation content is transmitted to the user; and a result output and notification unit for outputting and notifying the user and the doctor of the bio information, the blood pressure signal light information, the specific hypertension information, and the context information.

In this aspect, the hypertension monitoring and notification device may further comprise a dine-out index and exercise index calculation unit for calculating a dine-out index and an exercise index from a real-time position of the user and the weather and health information.

In this aspect, the data gain and storage unit may comprise a bio information database part and a context database part for collecting and storing the bio information and the context data from a personal health record device; and a weather and health database part for storing the weather and health information received from a weather station terminal.

In this aspect, the user context deduction and analysis unit may comprise a context information deduction part for deducing the context information regarding rising time, whether there was urination, and whether there was exercise or a meal taken, from the bio information and the context data; and a user context analysis part for determining whether a blood pressure measurement time is within a predetermined time after rising, is after urination, and is just after exercising or a meal, based on the deduced context information and the context information of the user input via a portable user terminal, so as to analyze context necessary to measure the hypertension.

In this aspect, the specific hypertension analysis unit may comprise a morning hypertension analysis part for analyzing morning hypertension where there is high blood pressure in early morning after rising; and a white-coat hypertension analysis part for analyzing white-coat hypertension in which a blood pressure measured by the doctor is higher by at least a predetermined value than home blood pressure or daytime blood pressure.

In this aspect, in the morning hypertension analysis part, morning blood pressure data may be obtained and a mean morning blood pressure value may be calculated and compared with a hospital blood pressure, so that in a case where the mean morning blood pressure value exceeds the hospital blood pressure, it is determined to be morning hypertension, and a case where the mean morning blood pressure value is not more than the hospital blood pressure it is determined to be normal blood pressure.

In this aspect, in the white-coat hypertension analysis part, home blood pressure data may be obtained and a mean home blood pressure value may be calculated and compared with a hospital blood pressure, so that in a case where a sum of the mean home blood pressure value and the predetermined value is not more than the hospital blood pressure, it is determined to be white-coat hypertension, and a case where a sum of the mean home blood pressure value and the predetermined value exceeds the hospital blood pressure, it is determined to be normal blood pressure.

In this aspect, the blood pressure grade analysis unit may comprise a blood pressure change and a doctor recommendation part for the doctor determining a blood pressure grade by using the analyzed context information thus changing the blood pressure grade via a doctor terminal, and for making a recommendation content to transmit it to a portable user terminal; and a blood pressure signal light analysis part for deducing the blood pressure signal light information based on the bio information and the determined blood pressure grade.

In this aspect, the result output and notification unit may transmit rising and sleeping time information, re-measurement and blood pressure measurement suggestions, the dine-out index and the exercise index, a specific hypertension determination notification message, and a notification message about a patient suspected of having morning hypertension, in order to accurately measure the blood pressure.

In this aspect, the result output and notification unit may transmit rising and sleeping time information, re-measurement and blood pressure measurement suggestions, the dine-out index and the exercise index, a specific hypertension determination notification message, and a notification message about a patient suspected of having morning hypertension, in order to accurately measure the blood pressure.

### Advantageous Effects of Invention

According to the present invention, a hypertension monitoring and notification device based on context information enables more accurate measurement values to be obtained in consideration of the context of a user, and can reduce the health risk the user faces by means of caring for a specific patient and the life indexes, so that the user can receive accurate and specialized services as well as more customized services.

In particular, this device can provide context information when taking blood pressure measurements of a patient with hypertension so that the patient can be continuously cared for at home and a doctor can clinically utilize the data measured at the home.

Furthermore, this device enables the context to be analyzed so that the user complies with measurement suggestions thus providing recommendation and notification services, and also enables the classification of a patient suspected of having specific hypertension who is difficult to diagnose in a hospital so that a doctor can be notified of it.

Furthermore, the doctor can confirm statistical information about the significant blood pressure data, and the user is well aware of the accurate guidelines for measuring blood pressure thus reducing the generation of unreliable blood pressure data, and can experience more specific and customized counseling from the doctor upon entering a hospital.

### BRIEF DESCRIPTION OF DRAWINGS

FIG. 1 is a block diagram showing a system comprising a hypertension monitoring and notification device based on context information according to the present invention;

FIG. 2 is a flowchart showing the operation of a normal blood pressure measurement algorithm using the hypertension monitoring and notification device based on context information according to the present invention;

FIG. 3 is a flowchart showing the operation of a blood pressure measurement algorithm depending on context using the hypertension monitoring and notification device based on context information according to the present invention;

FIG. 4 is a flowchart showing the operation of a morning hypertension measurement algorithm using the hypertension monitoring and notification device based on context information according to the present invention; and

FIG. 5 is a flowchart showing the operation of a white-coat hypertension measurement algorithm using the hypertension monitoring and notification device based on context information according to the present invention.

### MODE FOR THE INVENTION

Hereinafter, a hypertension monitoring and notification device based on context information according to the present invention is described with reference to the appended drawings.

In order to monitor the patient who is suffering from hypertension, a customized hypertension measurement algorithm based on blood pressure measurement suggestions taken under various contexts, and clinical standards for specific hypertension was designed in the present invention.

Clinical Standard of Hypertension Measurement

Hypertension is defined as shown in Table 1 below according to the seventh report (JNC7, 2003) of the Joint National Committee on the prevention, detection, evaluation and treatment of high blood pressure.

TABLE 1

| | | |
|---|---|---|
| Blood Pressure (BP) | | |
| Category | Systolic BP (mmHg) | Diastolic BP (mmHg) |

| | |
|---|---|
| Normal BP | 120 or less and 80 or less |
| Pre-hypertension | 120~139 or 80~89 |
| Stage 1 hypertension | 140~159 or 90~99 |
| Stage 2 hypertension | >=160 or >=100 |

The blood pressure is different whenever the heart beats, in the morning and evening depending on the season, upon sleeping and rising, upon standing up, or in other contexts. In particular, there is a great difference in blood pressure when in and outside of a medical office, and thus the truth about hypertension and the regulation or not thereof may be ascertained by measuring 24 hr ambulatory blood pressure or measuring the blood pressure at home.

In order to accurately measure blood pressure in the present invention, measurement suggestions under various contexts are summarized in Table 2 below. When the measured blood pressure is higher or lower than the normal level, the following contexts are checked and measurements are performed again.

TABLE 2

| Measurement ContextAfter | MeasurementTwo BP |
|---|---|
| measuring BP | measurements at intervals of 2 min |
| Morning | Measuring BP within 1 hr after rising, after |
| | urination, before taking medication, before |
| | breakfast |
| Evening | Measuring BP after stabilization for 1~2 min |
| | before sleeping, before taking medication, |
| | after urination, but never just after a meal |
| After exercising | Measuring BP 1~2 hr later |
| Upon smoking or having | Measuring BP 30 min later |
| coffee | |

Self blood pressure measurement is carried out within 1 hr after rising or after urination in the morning and after stabilization before sleeping in the evening, and home blood pressure measured at home is approximately lower by an average of 12/7 mmHg than the clinical blood pressure measured in a medical office. Based on the differences between the clinical blood pressure and the home blood pressure, hypertension may be classified into white-coat hypertension and masked hypertension. In addition, there are other types of specific hypertension, such as nighttime hypertension and morning hypertension.

Nighttime hypertension indicates that the blood pressure reaches a minimum at 2 AM while slowly dipping due to increasing activity of parasympathetic nerves and the decreasing activity of sympathetic nerves upon sleeping, and then increases again from 5 AM. In the case where the nighttime blood pressure is decreased by less than 10% compared to the daytime blood pressure is referred to as ‘nondipper’, and the risk of myocardial ischemia and ischemic stroke is higher in an extreme dipper (an overdipper) who has nighttime blood pressure that excessively decreases by 20% or more.

Morning hypertension means that the blood pressure in the early morning after rising is high. When the morning blood pressure remarkably increases, myocardial infarction, stroke, sudden death, and kidney injury frequently occur, and thus the detection and treatment of morning hypertension are regarded as important and the selection of medications that do not affect the 24 hr circadian pattern and drop the blood pressure for 24 hrs or that reduce an increase in the blood pressure in the morning is required.

White-coat hypertension means that the blood pressure measured by a doctor in a medical office is at least 10 mmHg higher than the home blood pressure or the daytime blood pressure and the frequency of white-coat hypertension is about 20˜30% of essential hypertension, and is higher in a female, a youth, a lightweight person, and a person with a short history of hypertension. Also, patients with white-coat hypertension should be sufficiently tested for whether they are suffering from metabolic diseases or from internal organ damage.

Masked hypertension means that the clinical blood pressure is normal but the daytime ambulatory blood pressure or the home blood pressure is high, and a youth, a smoker, an excessive alcohol consuming drinker, a person having abdominal obesity, a person having exercise insufficiency, and a person having high stress are highly attacked thereby and have an inferior prognosis to a normal person and thus the mass of the left ventricle and hypertrophy of the carotid artery become significant and the expansion of the main artery is negatively impacted resulting in high cardiovascular morbidity and mortality.

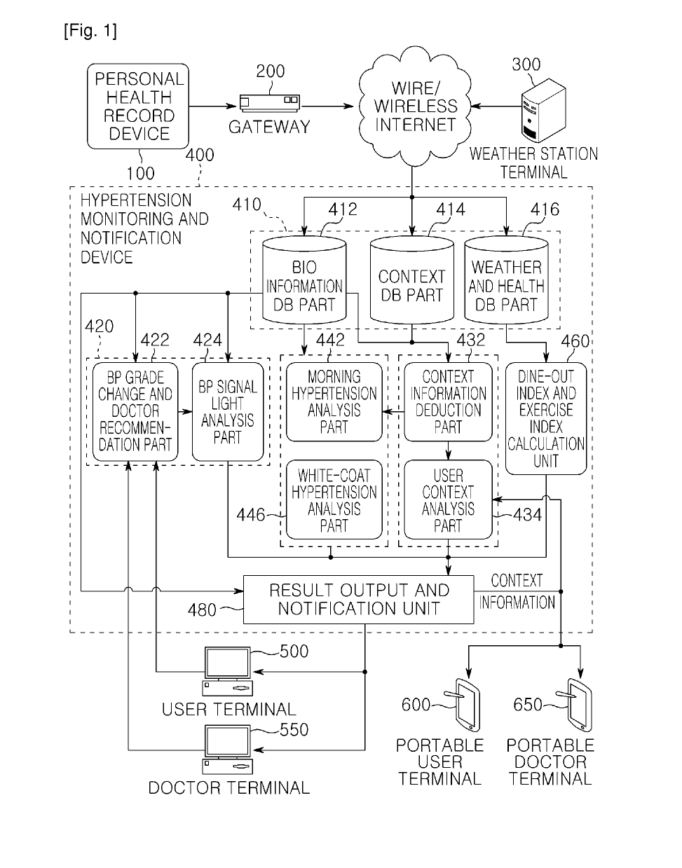

FIG. 1 is a block diagram showing a system comprising a hypertension monitoring and notification device 400 based on context information according to the present invention, which specifically comprises a personal health record device 100, a gateway 200, a weather station terminal 300, a hypertension monitoring and notification device 400, a user terminal 500 and a doctor terminal 550, and a portable user terminal 600 and a portable doctor terminal 650.

The hypertension monitoring and notification device 400 comprises a data gain and storage unit 410, a blood pressure grade analysis unit 420, a user context deduction and analysis unit 430, a specific hypertension analysis unit 440, a dine-out index and exercise index calculation unit 460, and a result output and notification unit 480.

The data gain and storage unit 410 includes a bio information database (DB) part 412, a context DB part 414 and a weather and health DB part 416, and the user context deduction and analysis unit 430 includes a context information deduction part 432 and a user context analysis part 434. The specific hypertension analysis unit 440 includes a morning hypertension analysis part 442 and a white-coat hypertension analysis part 446, and the blood pressure grade analysis unit 420 includes a blood pressure grade change and doctor recommendation part 422 and a blood pressure signal light analysis part 424.

With reference to FIG. 1, the operation of the system comprising the hypertension monitoring and notification device 400 based on context information according to the present invention is described below.

The bio signal of a patient is measured using a mounting/attaching device at home and the measured data is transmitted to the hypertension monitoring and notification device 400 via the gateway 200 and analyzed. The analyzed information is fed back to the user in the form of health information, notification, and recommendation via the web or the UI (user interface) of a mobile service, and the doctor utilizes it as basic information for managing the health of a patient via the doctor UI of the web service and may perform diagnosis and counseling.

Specifically, the bio information of the user and the context data are collected via the gateway 200 from the personal health record device 100, and are stored in the bio information DB part 412 and the context DB part 414 of the hypertension monitoring and notification device 400 via the Internet network. Furthermore, weather and health information are received from the weather station terminal 300 and stored in the weather and health DB part 416.

The context information deduction part 432 deduces context information including the ‘rising time’ and so on using the data of the bio information DB part 412 and the context DB part 414, and the user context analysis part 434 analyzes the context necessary for measuring hypertension including ‘measuring blood pressure within 1 hr after rising’ based on the context information deduced from the context information deduction part 432 and the context information input by a user via the portable user terminal 600.

The specific hypertension analysis unit 440 analyzes specific hypertension using the information of the bio information DB part 412 and the context information deduction part 432, and the dine-out index and exercise index calculation unit 460 calculates the dine-out index and the exercise index from the real-time position of a user and the data of the weather and health DB part 416.

The blood pressure grade change and doctor recommendation part 422 allows a doctor to change the blood pressure grade of a patient by menas of the doctor terminal UI and makes content about the doctor's recommendation which is to be transferred to the patient, and the blood pressure signal light analysis part 424 deduces a blood pressure signal light value of a user based on the data of the bio information DB part 412 and the blood pressure grade determined by the blood pressure grade change and doctor recommendation part 422.

The result output and notification unit 480 outputs the blood pressure signal light information, the specific hypertension content, the dine-out/exercise index, and the measurement context information resulting from measuring the bio signal, using the user terminal 500 and the doctor terminal 550, and notification services are provided to the user and the doctor based on the specific hypertension information and the information whose context was analyzed using the portable user terminal 600 and the portable doctor terminal 650.

Hypertension Monitoring and Notification Service Based on Context Information

1. The Normal Blood Pressure Measurement Algorithm

In the present invention, the hypertension monitoring and notification service is provided by checking the measurement context of a user and analyzing the bio measurement value using a system. In the case where there is an abnormal problem, the notification service provides re-measurement recommendation and measurement suggestions. Also, the graph and the health condition based on the measured values are monitored by the user and the doctor.

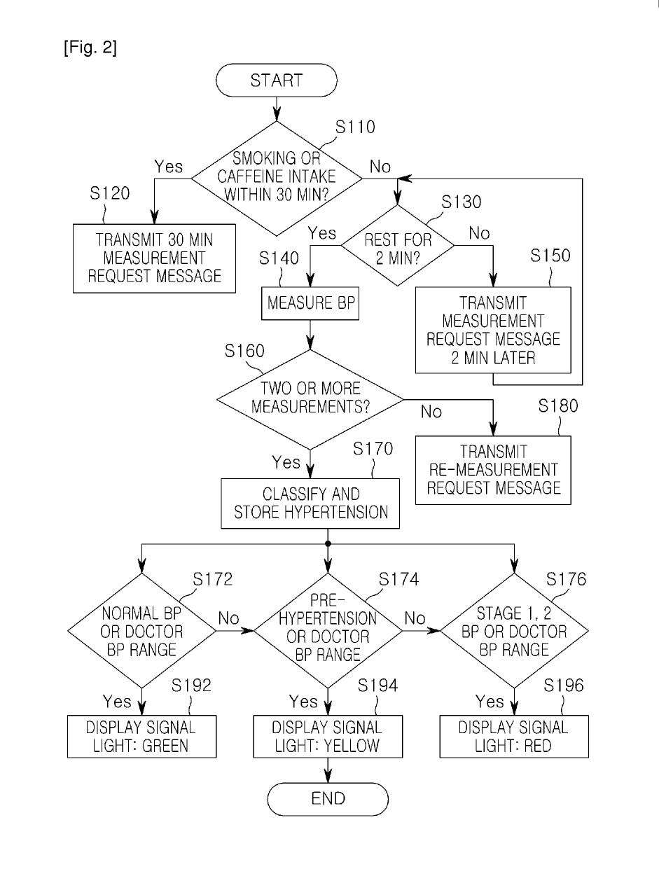

FIG. 2 is a flowchart showing the operation of a normal blood pressure measurement algorithm using the hypertension monitoring and notification device 400 based on context information according to the present invention.

With reference to FIGS. 1 and 2, the operation of the normal blood pressure measurement algorithm using the hypertension monitoring and notification device 400 based on context information according to the present invention is described below.

Whether one smoked or imbibed caffeine 30 min before measuring blood pressure and whether there was 2 min of rest or not are checked, and measurement suggestions are provided as notification services, and the accurately measured blood pressure value is received and then monitored via the blood pressure grade of a user and the blood pressure signal light based on the hypertension standards of Table 1.

Specifically, whether a user smoked or imbibed caffeine within 30 min before measuring the blood pressure is determined by the hypertension monitoring and notification device 400 (S110). In the case where the user smoked or imbibed caffeine within the 30 min, this fact is inputted to the portable user terminal 600, and the hypertension monitoring and notification device 400 receives it and controls the transmission of a 30 min measurement request message such as ‘please measure the blood pressure again 30 min later because smoking or caffeine intake affects blood pressure’ by means of the result output and notification unit 480 (S120).

In the case where the user neither smoked nor imbibed caffeine within 30 min before measuring the blood pressure, whether the user rested for 2 min is checked using the portable user terminal 600 (S130).

In the case where the user has rested for 2 min, the blood pressure is measured (S140), whereas in the case where the user did not rest for 2 min, the hypertension monitoring and notification device 400 controls the transmission of a measurement request message 2 min later such as ‘please measure the blood pressure after stabilization for 2 min’ by means of the result output and notification unit 480 (S150).

Whether the number of blood pressure measurements are two or more is determined by the hypertension monitoring and notification device 400 (S160). Upon two or more measurements, the classification of the type of hypertension is made depending on the systolic blood pressure and the diastolic blood pressure and stored (S170). On the other hand, upon less than two measurements, the hypertension monitoring and notification device 400 controls the transmission of a re-measurement request message such as ‘please measure the blood pressure again 2 min later’ by means of the result output and notification unit 480 (S180).

In the following, ‘doctor blood pressure’ is the standard blood pressure of a specific patient set by a doctor. The hypertension monitoring and notification device 400 allows the blood pressure signal light to be green when the measured blood pressure is in the range of normal blood pressure or the doctor blood pressure (S192), and the blood pressure signal light to be yellow when the measured blood pressure is in the range of pre-hypertension or the doctor blood pressure (S194), and the blood pressure signal light to be red when the measured blood pressure is in the range of stage 1 hypertension, stage 2 hypertension or the doctor blood pressure (S196).

2. The Blood Pressure Measurement Algorithm Depending on Context

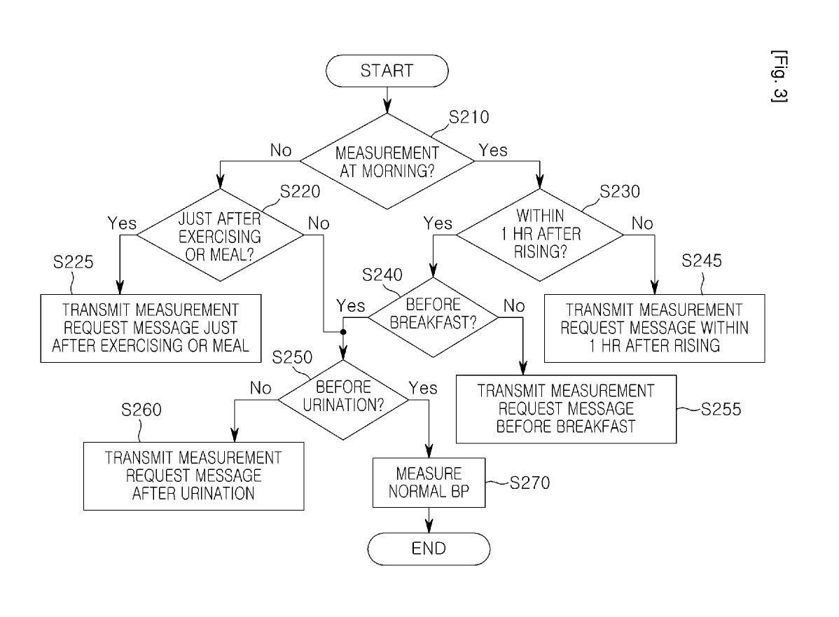

FIG. 3 is a flowchart showing the operation of a blood pressure measurement algorithm depending on context using the hypertension monitoring and notification device 400 based on context information according to the present invention.

With reference to FIGS. 1 and 3, the operation of the blood pressure measurement algorithm depending on context using the hypertension monitoring and notification device 400 based on context information according to the present invention is described below.

In the present invention, the time of measurement and whether it is before or after rising are checked using a smart device and the context information is obtained from the user, so that the measurement context is analyzed.

In the blood pressure measurement depending on context, in the case where the measurement time is in the morning, whether it is within 1 hr after rising is checked, and also whether it is before breakfast or after urination is checked, so that the blood pressure can be accurately measured.

In the case where the measurement time is not in the morning, whether it is just after exercising or a meal is checked and whether it is after urination is checked, so that a notification service about the accuracy of the blood pressure measurement is provided to the user.

Whether the measurement time is in the morning is determined by the hypertension monitoring and notification device 400 (S210). In the case where the measurement time is in the morning, whether it is within 1 hr after rising is determined (S230), whereas in the case where the measurement time is not in the morning, whether it is just after exercising and a meal is determined (S220).

In the case where the measurement time is within 1 hr after rising at S230, whether it is before breakfast is determined (S240). In the case where the measurement time is not within 1 hr after rising, the hypertension monitoring and notification device 400 controls the transmission of a measurement request message within 1 hr after rising such as ‘please measure the blood pressure within 1 hr after rising’ by means of the result output and notification unit 480 (S245).

In the case where the measurement time is just after exercising or a meal at S220, the hypertension monitoring and notification device 400 controls the transmission of a measurement request message just after exercising or a meal such as ‘please measure the blood pressure again 1 hr later’ by means of the result output and notification unit 480 (S225). In the case where the measurement time is not just after exercising or a meal, whether it is after urination is determined (S250).

In the case where the measurement time is before breakfast at S240, whether it is after urination is determined (S250). In the case where the measurement time is after breakfast, the hypertension monitoring and notification device 400 controls the transmission of a measurement request message before breakfast such as ‘please measure the blood pressure before breakfast tomorrow’ by means of the result output and notification unit 480 (S255).

In the case where the measurement time is after urination at S250, the normal blood pressure is measured (S270). When the measurement time is before urination, the hypertension monitoring and notification device 400 controls the transmission of a measurement request message after urination such as ‘please measure the blood pressure after urination’ by means of the result output and notification unit 480 (S260).

3. The Morning Hypertension Measurement Algorithm

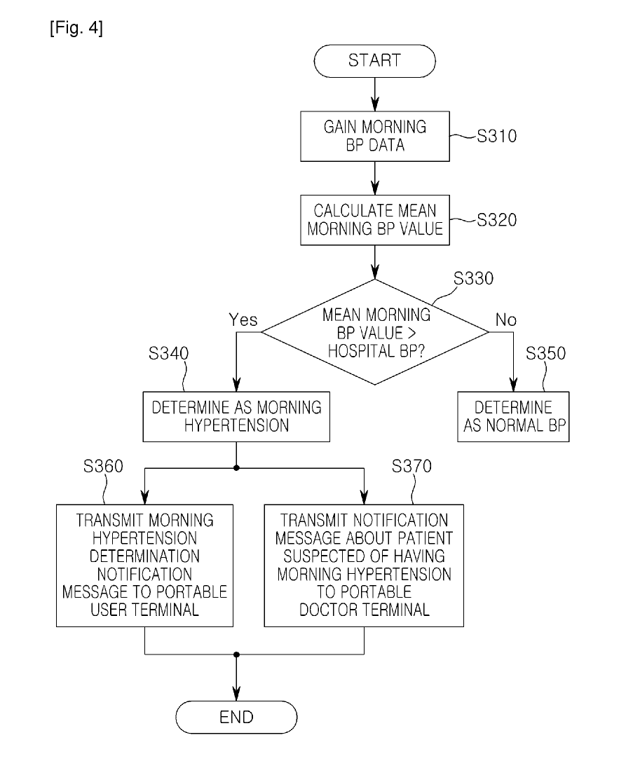

FIG. 4 is a flowchart showing the operation of a morning hypertension measurement algorithm using the hypertension monitoring and notification device 400 based on context information according to the present invention.

With reference to FIGS. 1 and 4, the operation of the morning hypertension measurement algorithm using the hypertension monitoring and notification device 400 based on context information according to the present invention is described below.

Morning hypertension means that there is high blood pressure in the early morning after rising. The case where the blood pressure drastically increases in the morning may frequently cause myocardial infarction, stroke, sudden death and kidney injury, and thus the blood pressure should be continuously monitored so that it can be managed. Hence, in the present invention, the morning blood pressure value is determined via blood pressure measurement monitoring depending on context, so that monitoring and notification services for analyzing the morning hypertension of a suspected patient are provided to a user.

Specifically, morning blood pressure data is obtained (S310) and a mean morning blood pressure value is calculated (S320). Whether the mean morning blood pressure value exceeds the hospital blood pressure is determined by the hypertension monitoring and notification device 400 (S330). In the case where it exceeds the hospital blood pressure, it is determined to be morning hypertension (S340), whereas in the case where it is not more than the hospital blood pressure, it is determined to be normal blood pressure (S350).

After S340, a morning hypertension determination notification message is transmitted to the portable user terminal 600 (S360), and a notification message about the patient suspected of having morning hypertension is transmitted to the portable doctor terminal 650 (S370).

b 4. The White-Coat Hypertension Measurement Algorithm

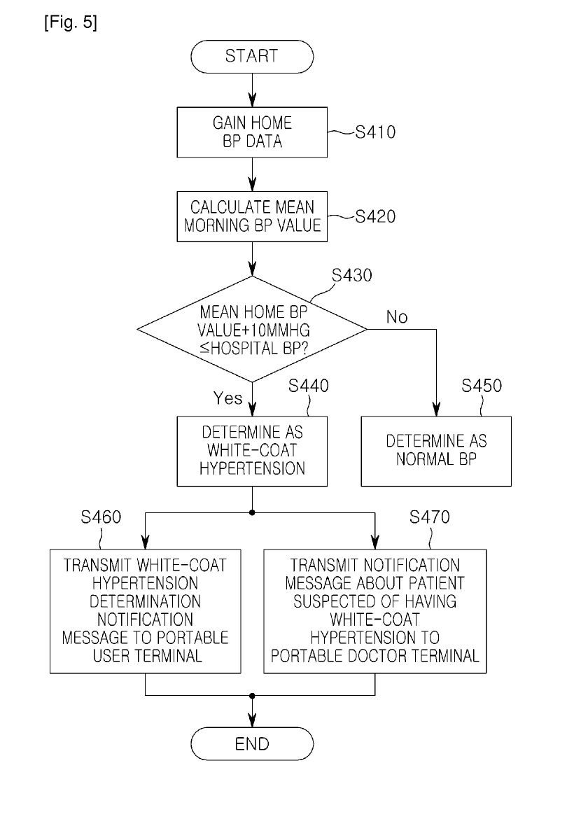

FIG. 5 is a flowchart showing the operation of a white-coat hypertension measurement algorithm using the hypertension monitoring and notification device 400 based on context information according to the present invention.

With reference to FIGS. 1 and 5, the operation of the white-coat hypertension measurement algorithm using the hypertension monitoring and notification device 400 based on context information according to the present invention is described below.

White-coat hypertension means that the blood pressure measured by a doctor in a medical office is at least 10 mmHg higher than the home blood pressure or the daytime blood pressure, and the frequency thereof is about 20˜30% of essential hypertension and is higher in a female, a youth, a lightweight person, and a person with short history of hypertension. Because whether a patient with white-coat hypertension suffers from metabolic diseases or has internal organ damage should be sufficiently examined, a monitoring service able to compare the home measurement results with hospital measurement results is required.

Specifically, home blood pressure data is obtained (S410) and a mean home blood pressure value is calculated (S420). Whether the sum of the mean home blood pressure value and 10 mmHg is not more than the hospital blood pressure is determined by the hypertension monitoring and notification device 400 (S430). In the case where it is not more than the hospital blood pressure, it is determined to be white-coat hypertension (S440), whereas in the case where it exceeds the hospital blood pressure, it is determined to be normal blood pressure (S450).

After S440, a white-coat hypertension determination notification message is transmitted to the portable user terminal 600 (S460), and a notification message about the patient suspected of having white-coat hypertension is transmitted to the portable doctor terminal 650 (S470).

5. Providing Dine-Out and Exercise Indexes

In order to manage the health of a patient with hypertension, diet and exercise are regarded as important. However, outdoor exercise in an environment contaminated by weather events or industrialization may cause the patient to be exposed to not only hypertension but also other diseases. Hence, in the present invention, the dine-out and exercise indexes are divided into high, medium, and low using a life weather index and a health weather index provided from a weather station. The exercise index and the dine-out index as shown in Tables 3 and 4 below are provided to the user to help manage the health of the patient.

TABLE 3

| | March~November, | | | | | |
|---|---|---|---|---|---|---|
| | June~September | | | December~February | | |
| | Exercise Index | | | | | |
| | | Index | | | Index | Freezing |
| | | rela- | Heat | Effective | rela- | burst |
| | UV index | tion | index | temp. | tion | index |
| High | low | and | low | | | |
| Medium | low | and | medium | interest | and | medium |
| Low | danger, | or | very | caution, | or | very |
| | very high, | | high, | warning, | | high, |
| | high, | | high | danger | | high |
| | medium | | | | | |

TABLE 4

| | March~November | | | December~February | | |
|---|---|---|---|---|---|---|
| | Dine-out Index | | | | | |
| | Food | Index | | | Index | Freezing |
| | poisoning | rela- | | Effective | rela- | burst |
| | index | tion | UV index | temp. | tion | index |
| High | interest | and | low | interest | and | low |
| Medium | interest | and | medium | interest | and | medium |
| Low | danger, | or | very | caution, | or | very |
| | warning | | high, | warning, | | high, |
| | | | high, | danger | | high |
| | | | danger | | | |

6. Hypertension Notification Service

In the present invention, the notification service is provided after analyzing the measured bio signal and the context information so that measurement recommendation notification, specific hypertension notification, and measurement context notification are provided by SMS (Short Message Service) or using a popup window.

The measurement recommendation notification service shown in Table 5 below provides the blood pressure measurement notification to a user by the user inputting the rising time and the sleeping time and analyzing the rising using a smart bed.

TABLE 5

| | | | | | Method of |
|---|---|---|---|---|---|
| | | | | | gaining recommendation |
| Service | Time | Means | Target | Period | time information |
| Measurement | within 30 min | SMS | Pppatient, General | Everyday | Inputting by |
| recommendation | after | | person | | a user, |
| | rising | | | | Device |
| | 30 min | | | | |
| | before | | | | |
| | sleeping | | | | |

The specific hypertension notification service shown in Table 6 below notifies a service user and a doctor, of a patient suspected of having nighttime, morning, white-coat and masked hypertension, provided that in the present invention only the morning hypertension and white-coat hypertension prediction and notification services are supplied because of the limitations of the device and system and depending on clinical needs.

TABLE 6

| Service | Means | Target | Period | Detailed Service |
|---|---|---|---|---|
| Specific hypertension | SMS, | User, | All | Notification about patient |
| notification | Terminal | Doctor | times | suspected of having nighttime |
| | | | | hypertension |
| | | | | Notification about patient |
| | | | | suspected of having morning hypertension |
| | | | | Notification about patient |
| | | | | suspected of having white-coat |
| | | | | hypertension |
| | | | | Notification about patient |
| | | | | suspected of having masked hypertension |

The measurement context notification service shown in Table 7 below recommends re-measurement and blood pressure measurement suggestions depending on the measurement context based on the above hypertension measurement guideline.

TABLE 7

| | | | | |
|---|---|---|---|---|
| | | | | Detailed ServiceNotification |
| ServiceMeasurement | | TargetPatient, | | depending on whether there |
| context notification | MeansSMS, | General | PeriodBefore | is smoking or caffeine intake |
| service | Terminal | person | or after measurement | 30 min prior |

Notification depending on
whether there is a 2 min rest
Blood pressure re-
measurement notification
Notification depending on
after exercising or a meal
Notification depending on
whether measurement is
performed within 1 hr after
rising
Notification depending on
breakfast
Notification depending on
after urination or depending
on taking medicine

The hypertension monitoring and notification device based on context information according to the present invention provides the context information upon taking blood pressure measurements so that the hypertension patient can be continuously cared for at home and the data measured at home can be clinically used by a doctor. Thereby, the context is analyzed so that the user complies with the measurement suggestions,recommendation and notification services can be provided, and the patient suspected of having specific hypertensionthat is difficult to be diagnosed in a hospital is classified, and a doctor is notified.

Thus, a doctor can confirm the statistical information of significant blood pressure data, and a user is well aware of the accurate guidelines regarding blood pressure measurement, thus reducing the generation of unreliable blood pressure data, and attaining more specific and customized counseling from the doctor upon entering a hospital.

Furthermore, more accurately measured values may be obtained in consideration of the context of a user and the health risk of a user may be reduced via the management of specific patients and life indexes, thus providing the user with more accurate, detailed, and customized services.

1. A hypertension monitoring and notification device based on context information, comprising:
a data gain and storage unit for collecting bio information of a user and context data, and storing weather and health information;
a user context deduction and analysis unit for deducing context information of the user from the bio information and the context data to analyze context necessary to measure hypertension;
a specific hypertension analysis unit for analyzing specific hypertension using the bio information and the context information;
a blood pressure grade analysis unit for enabling a doctor to determine a blood pressure grade via the analyzed context information so that blood pressure signal light information is deduced and a recommendation content is transmitted to the user; and
a result output and notification unit for outputting and notifying the user and the doctor of the bio information, the blood pressure signal light information, specific hypertension information, and the context information.

2. The hypertension monitoring and notification device of claim 1, further comprising a dine-out index and exercise index calculation unit for calculating a dine-out index and an exercise index, from a real-time position of the user and the weather and health information.

3. The hypertension monitoring and notification device of claim 1, wherein the data gain and storage unit comprises:
a bio information database part and a context database part for collecting and storing the bio information and the context data from a personal health record device; and
a weather and health database part for storing the weather and health information received from a weather station terminal.

4. The hypertension monitoring and notification device of claim 1, wherein the user context deduction and analysis unit comprises:
a context information deduction part for deducing the context information regarding rising time, whether there was urination and whether there was exercise or a meal taken, from the bio information and the context data; and
a user context analysis part for determining whether a blood pressure measurement time is within a predetermined time after rising, is after urination, and is just after exercising or a meal, based on the deduced context information and the context information of the user input via a portable user terminal, so as to analyze context necessary to measure the hypertension.

5. The hypertension monitoring and notification device of claim 1, wherein the specific hypertension analysis unit comprises:
a morning hypertension analysis part for analyzing morning hypertension where there is high blood pressure in the early morning after rising; and
a white-coat hypertension analysis part for analyzing white-coat hypertension in which blood pressure measured by the doctor is higher by at least a predetermined value than home blood pressure or daytime blood pressure.

6. The hypertension monitoring and notification device of claim 5, wherein in the morning hypertension analysis part, morning blood pressure data is obtained and a mean morning blood pressure value is calculated and compared with a hospital blood pressure, so that where the mean morning blood pressure value exceeds the hospital blood pressure, it is determined to be morning hypertension, and where the mean morning blood pressure value is not more than the hospital blood pressure, it is determined to be normal blood pressure.

7. The hypertension monitoring and notification device of claim 5, wherein in the white-coat hypertension analysis part, home blood pressure data is obtained and a mean home blood pressure value is calculated and compared with a hospital blood pressure, so that where a sum of the mean home blood pressure value and the predetermined value is not more than the hospital blood pressure, it is determined to be white-coat hypertension, and where a sum of the mean home blood pressure value and the predetermined value exceeds the hospital blood pressure, it is determined to be normal blood pressure.

8. The hypertension monitoring and notification device of claim 1, wherein the blood pressure grade analysis unit comprises:
a blood pressure change and doctor recommendation part for the doctor determining a blood pressure grade by using the analyzed context information thus changing the blood pressure grade via a doctor terminal, and for making a recommendation content to transmit it to a portable user terminal; and
a blood pressure signal light analysis part for deducing the blood pressure signal light information based on the bio information and the determined blood pressure grade.

9. The hypertension monitoring and notification device of claim 2, wherein the result output and notification unit transmits rising and sleeping time information, re-measurement and blood pressure measurement suggestions, the dine-out index and the exercise index, a specific hypertension determination notification message, and a notification message about a patient suspected of having morning hypertension, in order to accurately measure the blood pressure.
